(19) United States
(12) Patent Application Publication
(10) Pub. No.: US 2017/0209170 A1
(43) Pub. Date: Jul. 27, 2017

(54) CUSTOM NEEDLE GUIDE APPARATUS AND METHOD FOR MANUFACTURE IN A MEDICAL PROCEDURE

(71) Applicant: Harmonus Inc., Lowell, MA (US)

(72) Inventors: John Carl Magill, Woburn, MA (US); Peter Tia, Dracut, MA (US); Phillip John Marathakis, Cambridge, MA (US)

(21) Appl. No.: 15/418,292

(22) Filed: Jan. 27, 2017

Related U.S. Application Data
(60) Provisional application No. 62/287,637, filed on Jan. 27, 2016.

(51) Int. Cl. A61B 17/34; B29C 65/06; A61B 10/02

(52) U.S. Cl. CPC A61B 17/3403; A61B 10/0241; A61B 10/0266; B29C 65/0672; A61B 2010/0225; A61B 2010/045

(57) ABSTRACT

A sterile needle guide for use during a biopsy procedure includes a planar plate having at least one tubular needle guide extending there through. Needle guide may be manufactured from a kit having plurality of guide tubes each paired with a drill bit. In practice, a drill bit and guide tube are coupled to a drilling machine and positioned relative to a planar blank at a location derived from previously recorded or real-time imaging data. Drilling through the plate causes the guide tube to extend through the plate and become thermally fused therewith. In one embodiment, a guide tube includes multiple needle guide passages enabling multiple, clustered insertion points in a single area from a single guide tube.

### BACKGROUND OF THE DISCLOSURE

Prostate cancer is the second leading cause of cancer death in the U.S. Over 225,000 cases of cancer were diagnosed in 2014 with almost 30,000 deaths. Prostate cancer is treatable, if properly diagnosed. The initial screen to identify men with prostate cancer is the level of prostate-specific antigen PSA in blood. These men are often referred for a core biopsy in which samples of the prostate are excised and evaluated by a pathologist to determine if cancerous cells are present. There is no information in a PSA blood test to determine where cancerous tissue might be found in the prostate. A 12-core sampling distributed over the prostate has become the accepted method to determine if cancer is present. The procedure is typically conducted using a trans-rectal ultrasound to visualize the needle location, and the needle is typically inserted through the lining of the rectum to reach the prostate. The trans-rectal ultrasound-guided procedure requires a large number of needle insertions, requires high doses of antibiotic prophylaxis, does not make the suspicious region easily visible, and provides no means of recording sample locations for future reference.

Accordingly, need exists for reducing the number of needle insertions in a prostate biopsy procedure and the need for antibiotics by avoiding accessing the prostate through the rectum.

A further need exists for a technique which can provide a precise recording of the needle tip location during a biopsy.

An even further need exists for a patient-specific disposable tool for directing needle entry during a medical procedure, such as an MRI-guided prostate biopsy.

### SUMMARY OF THE DISCLOSURE

Disclosed is a customizable needle guide for directing needle entry during a medical procedure, such as an MRI-guided prostate biopsy. In embodiments, the needle guide comprises a plate with one or more tubes inserted into the plate at locations chosen based on patient position and MR images during a biopsy procedure.

Also disclosed is a method for making a patient-specific disposable tool for directing needle entry during a medical procedure such as an MRI-guided prostate biopsy. More specifically, disclosed is a method for making a needle guide to guide the needle while the patient remains in the MRI. The target locations can be identified in the reference frame of the scanner using initial images and pre-operative multi-parameter MRI, and trajectories can be selected. The needle guide would then be produced and used to complete the biopsy. The needle guide may be created by a method in which a hole is drilled in a plastic plate by friction and a guide tube is inserted and welded in place by friction welding. Fabrication of the needle guide is based on a rapid, precision machine for creating the guide from a sterile kit.

According to one aspect of the disclosure, a sterile needle guide for use during a biopsy procedure comprises a planar plate having at least one tubular needle guide extending there through.

According to another aspect of the disclosure, a kit for manufacturing a needle guide under sterile conditions comprises a plurality of guide tubes each having a drill bit paired therewith. In one embodiment, the kit may optionally further comprise a planar blank into which the guide tubes are embedded during the process.

According to yet another aspect of the disclosure, a system comprises in combination a cylindrical guide tube defining at least one needle guide passage extending therethrough, a drill bit coupled to the guide tube at a first end thereof, and an adapter cap for coupling one of the drill bit and guide tube to a source of motion.

According to still another aspect of the disclosure, a sterile needle guide for use during a biopsy procedure comprises a cylindrical guide tube body defining a plurality of needle guide passages extending therethrough and having a first end defined for coupling to a drill bit and a second end defined for coupling with an adapter.

According to yet another aspect of the disclosure, A kit for preparing a needle guide for biopsy procedures comprises: a cylindrical guide tube defining an interior needle guide passage extending therethrough, a drill bit coupled to the guide tube at a first end thereof, a planar plate formed of a material having a lower melting point than the drill bit; and an encapsulating structure defining a selectively accessible interior space in which the guide tube and planar plate are disposed. In one embodiment, the encapsulating structure is defined by a wall which is at least partially movable relative to another section of the encapsulating structure and which allows the guide tube to be re-positioned along three axes relative to the planar plate while remaining within the interior space of the encapsulating structure.

### DESCRIPTION THE DRAWINGS

FIG. 1 illustrates conceptually a guide for prostate biopsy in the bore of an MRI scanner;

FIG. 2 illustrates conceptually a needle guide template in accordance with the present disclosure;

FIG. 3 illustrates conceptually an apparatus for automatically moving a drill relative to a plate in accordance with the present disclosure;

FIG. 4 illustrates conceptually a side, cross-sectional view of kit and drill in accordance with the present disclosure;

FIGS. 5A-F illustrate conceptually a fabrication process used to generate a custom needle guide in accordance with the present disclosure;

FIGS. 6A-C illustrate conceptually interfaces between a non-sterile drill and sterile parts of the kit in accordance with the present disclosure in accordance with the present disclosure;

FIGS. 7A-C illustrate conceptually drill bit configurations in accordance with the present disclosure;

FIGS. 8A-B illustrate conceptually drill bit configurations in accordance with the present disclosure;

FIGS. 9A-D illustrate conceptually exploded, perspective views of drill bit, guide tube and/or adapter combinations in accordance with the present disclosure;

FIGS. 10A-B illustrate conceptually side and perspective views of a cylindrical needle guide in accordance with the present disclosure;

FIG. 11A illustrates conceptually an exploded, perspective views of a drill bit, guide tube and adapter combinations in accordance with the present disclosure;

FIG. 11B illustrates conceptually an exploded, perspective views of a kit in accordance with the present disclosure;

FIG. 11C illustrates conceptually a kit disposed inside an outer sterile package in accordance with the present disclosure;

FIGS. 12A-F illustrate conceptually a sterile fabrication process used to generate a custom needle guide from the a kit in accordance with the present disclosure;

FIGS. 13A-B illustrate conceptually exploded and side views of drill bit, guide tube and/or adapter CAP combinations in accordance with the present disclosure;

FIGS. 14A-D illustrate conceptually perspective, side, cut-away, and lateral views, respectively, of a kit in accordance with the present disclosure;

FIGS. 15A-B are views of another kit in accordance with the present disclosure;

FIG. 16 is a top view of the kit of FIGS. 14 and 15;

FIGS. 17 A-B illustrate conceptually the relationship of the kit of FIGS. 14 and 15 relative to a fabrication machine in accordance with the present disclosure,

FIGS. 18A-H illustrate conceptually the process sequence by which a fabrication machine is used to generate a custom needle guide utilizing kit of FIGS. 14 and 15;

FIG. 19 is a perspective view of another kit in accordance with the present disclosure;; and

FIGS. 20A-F illustrate conceptually the process sequence by which a fabrication machine is used to generate a custom needle guide utilizing kit of FIG. 19.

### DETAILED DESCRIPTION

According to one aspect of the disclosure, a method for quickly creating a customized sterile needle guide based on an MRI scan is disclosed. The customization procedure is performed while the patient is in the scanner, only a few minutes after the intra-operative scan that is used to design the template. There is not time to sterilize the parts after fabrication, so the needle guide template is produced under sterile conditions.

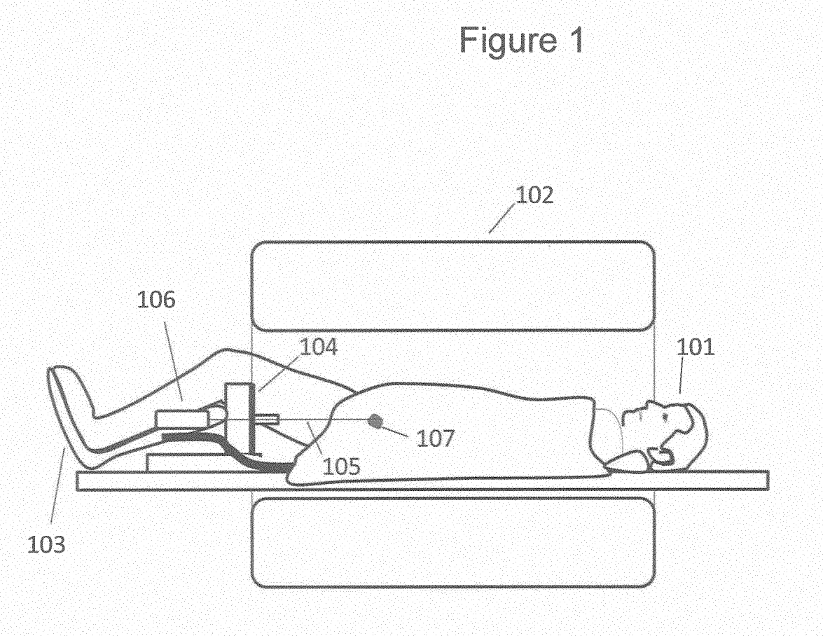

FIG. 1 illustrates conceptually the process for an MRI-guided trans-perineal approach using a fixed patient-specific template to guide the needle to targets. In the disclosed method, the patient 101 is placed in the MRI scanner 102 with his feet fixed in stirrups 103. A needle guide 104 created during the procedure after an initial scan of the patient is used to guide the biopsy needle 105 that is attached to the needle activator 106. The needle guide is constructed to permit the needle 105 to be inserted into a chosen location within the prostate 107. By accurately targeting MRI-visible regions suspected of containing cancer, the disclosed apparatus and technique will improve the ability to find life-threatening tumors and reduce the chance of unnecessarily treating low-risk cases compared to current methods.

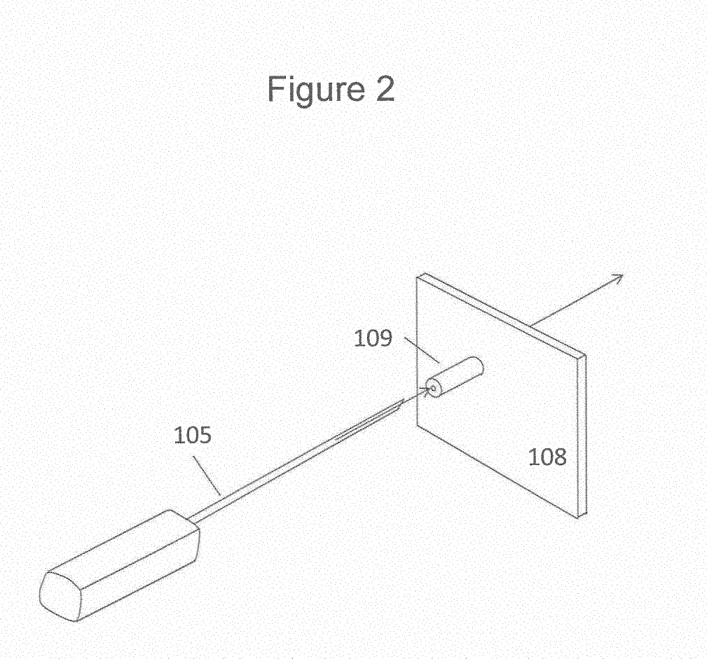

FIG. 2 illustrates conceptually a needle guide in accordance with the present disclosure. The needle guide comprises a flat plate 108 with one or more guide tubes 109 embedded therein. A biopsy needle 105 is inserted through guide tube 109 and into the perineum. The guide tubes may be color coded to match regions highlighted on a computer screen image. The guide tubes 109 are joined to the flat plate 108 at positions that correspond to the axis of the needle, when the guide is mounted in the scanner. In an illustrative embodiment, both the flat plate 108 and guide tubes 109 are made from a substantially rigid material such as natural or synthetic resins which can be manipulated for rapid manufacturer of the needle guide in accordance with the methods disclosed herein.

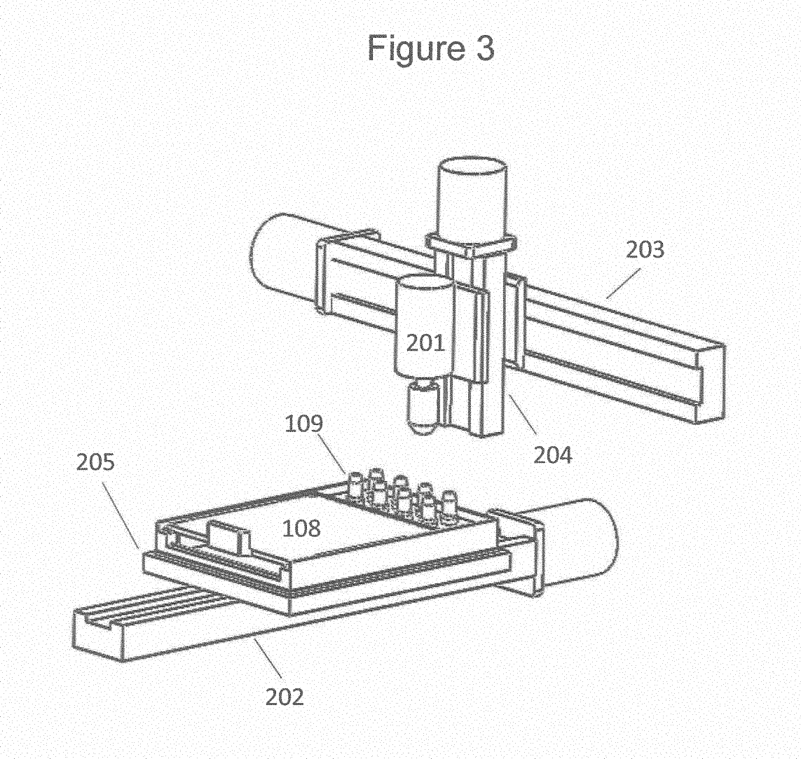

Referring to FIG. 3, a machine 200 for manufacturing a sterile needle guide during a medical procedure comprises a drill 201 and a system of automated actuators 202-204 that permit motion of the drill 201 relative to a stage 205 along three axes, so the guide tube 109 can be positioned with sufficient range over the plate 108 mounted on the stage and the drill can move along its own axis to create a hole in the plate. The machine 200 and, in particular, automated actuators 202, 203 and 204 may be implemented using any number of commercially available server motor actuated components which may be numerically controlled based on data derived from image data taken either prior to or during the medical procedure.

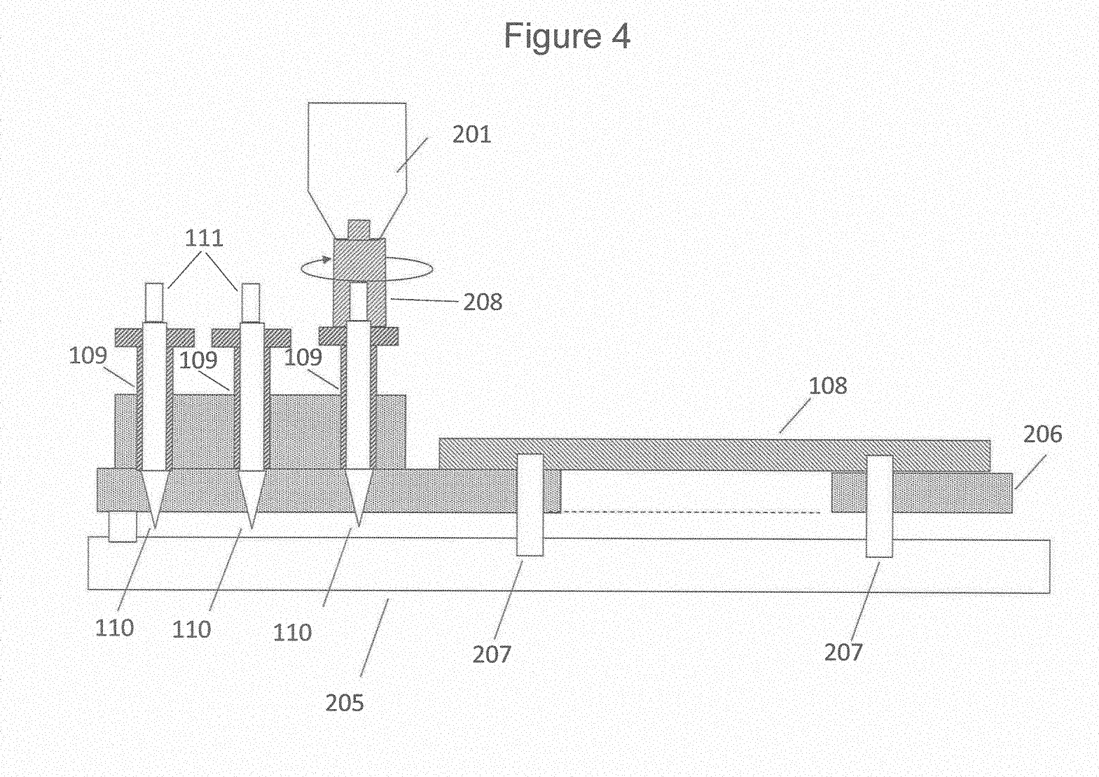

FIG. 4 illustrates conceptually a side, cross-sectional view of an exemplary implementation of a kit for creating the sterile needle guides. The kit 225 comprises several rigid guide tubes 109, e.g. cylinders made of thermoplastic, each having a friction-drilling bit 110, e. g., high-temperature plastic, contained therein and on a sterile tray 206. A sterile adapter 210 may be included in the kit to provide an interface between the drill motor 201 and a surface 211 on either the friction-drilling bits 110 or guide tubes 109. The purpose of the sterile adapter is to prevent transfer of contaminants or microorganisms from the drill motor or associated extensions to the bits or guide tubes. The sterile adapter may be attached manually to the drill motor or it may be removed from the kit and attached to the drill motor by automated means. The bits and guides are mated in pairs so that the guide tube cannot spin with respect to the bit, permitting the drill to spin both guide tube and bit when either is held in the adapter. A blank flat plate 108 may optionally be attached to tray 206 or may be provided separately. Guide features 207 align the plate to the stage 205 in the automated template-making machine 200.

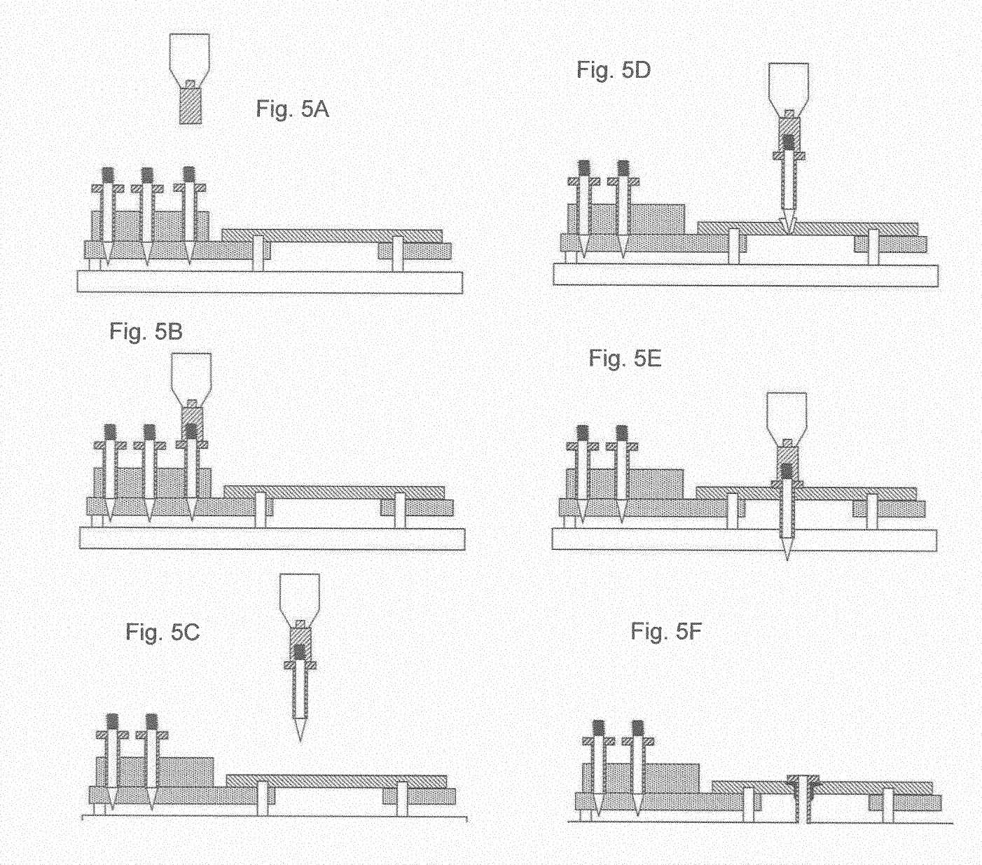

FIGS. 5A-F illustrate conceptually the process sequence by which the machine 200 is used to generate a custom guide from the disclosed kit. As noted, fabrication of the needle guide utilizes a rapid, precision machine for creating the guide from a sterile kit. In an illustrative sequence of images in FIGS. 5A-F, assume that the plate moves in two axes normal to the drill axis, though the same process can be accomplished by moving the motor itself. In FIG. 5A, the machine moves the plate such that one of the bit/guide tube pairs is directly under the drill motor with a sterile adapter. The drill moves down and attaches to the bit, as illustrated in FIG. 5B. This could be by mechanical threading or use of an electromagnetic collet. The drill is raised and the plate is moved so that the drill is directly over the point on the plate where the guide tube is to be installed, as illustrated in FIG. 5C. The drill spins to a high speed and descends so the bit tip contacts the template and begins to melt the plate locally by friction heating as illustrated in FIG. 5D. The bit continues to descend until the guide tube flange is against the plate top surface, as illustrated in FIG. 5E. The drill stops spinning the guide tube, allowing it to stop by friction, heating the surfaces of the guide tube and plate so they melt and fuse together. The drill releases the bit, as illustrated in FIG. 5F, allowing the bit to drop out of the guide tube. Note that the plate and guide tubes are never contacted by any part of the unsterile machine 200 or drill motor 201.

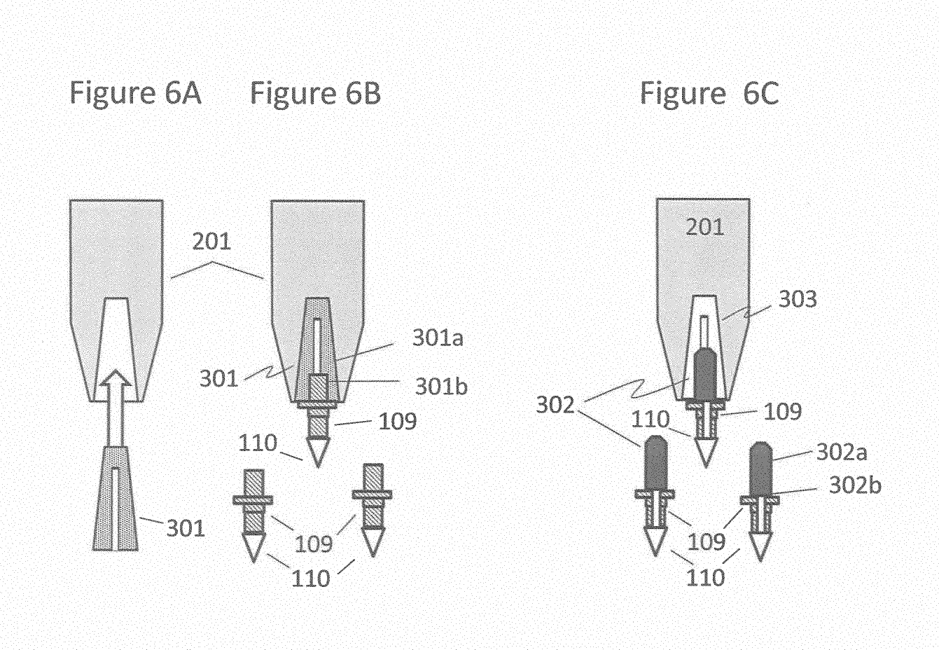

FIGS. 6A-C illustrate conceptually methods for manipulating sterile components, e.g., guide tubes and drill bits, with a non-sterile drill without contaminating the sterile components by employing a sterile adapter. Sterile adapters 301 or 302 each have at least a first exterior surface, 301a or 302a, respectively, that frictionally engages the drill 201 and may become contaminated upon contacting the drill as well as a second interior surface, 301b or 302b, respectively, that remains sterile and are used to contact the sterile components of the kit. Sterile adapter 301 may be implemented as a single integrally formed piece of semi-flexible material having an exterior surface with a profile which complements the features, e.g. a cavity or interior profile, of the drill bit or collet into which the sterile adapter is to be inserted and frictionally retained therein, as illustrated in FIGS. 6A-B. In embodiments, one end of the sterile adapter may include, features, e.g. a cavity or interior profile, which are designed to receive and frictionally retain an end of guide tubes 109 therein while the adapter 301 itself is attached to the drill 201. In one embodiment, as illustrated in FIG. 6C, an adapter 302 may be pre-fitted over a component e.g., guide tube 109 and bit 110, and attached to drill 201 with a non-sterile collet 303. The sterile adapters disclosed herein may be attached manually to a drill motor or may be removed from the sterile kit and attached to the drill motor by automated means. As noted previously, in embodiments, the sterile kit may include an adapter pre-fitted onto each of the guide to and drillbit combinations within the kit.

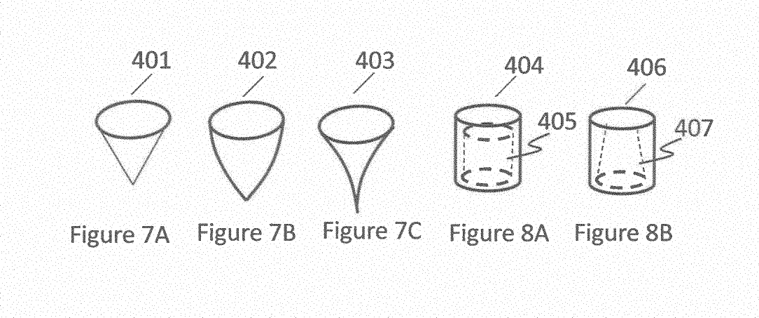

According to another aspect of the disclosure, a number of different drill bit and adapter configurations may be utilized to frictionally weld the guide tubes 109 to the plate 108. Referring to FIGS. 7A-C and 8A-B, a number of different drill bit configurations are illustrated. FIGS. 7A-C illustrate several generally conical shaped drill bit tips suitable for use with the disclosed embodiments. FIG. 7A illustrates the drill bit tip 401 having a generally conical shape with uniformly tapered sides. FIG. 7B illustrates the drill bit tip 402 having a generally conical shape with sides that taper non-uniformly to have an at least partially curved convex exterior profile. FIG. 5c illustrates a drill bit tip 403 having a generally conical shape with sides that taper non-uniformly to have an at least partially curved concave exterior profile. These generally conical shaped bits push material out of the way, penetrating the surface of plate 108 and gradually melting a larger diameter therein.

FIGS. 8A-B illustrate several generally cup shaped drill bit tips suitable for use with the disclosed embodiments. FIG. 8A illustrates a drill bit tip 404 having a generally cup shape characterized by a uniform diameter cavity 405, illustrated in phantom, extending at least partially through the interior thereof. FIG. 8B illustrates a drill bit tip 406 having a generally cup shape characterized by a tapered diameter cavity 407, illustrated in phantom, extending at least partially through the interior thereof. During the welding process, the generally cup-shaped drill bits 404 and 406 melt a ring along their respective outer diameters and capture the material excised from plate 108 and retain such material as a plug inside their respective interior cavities.

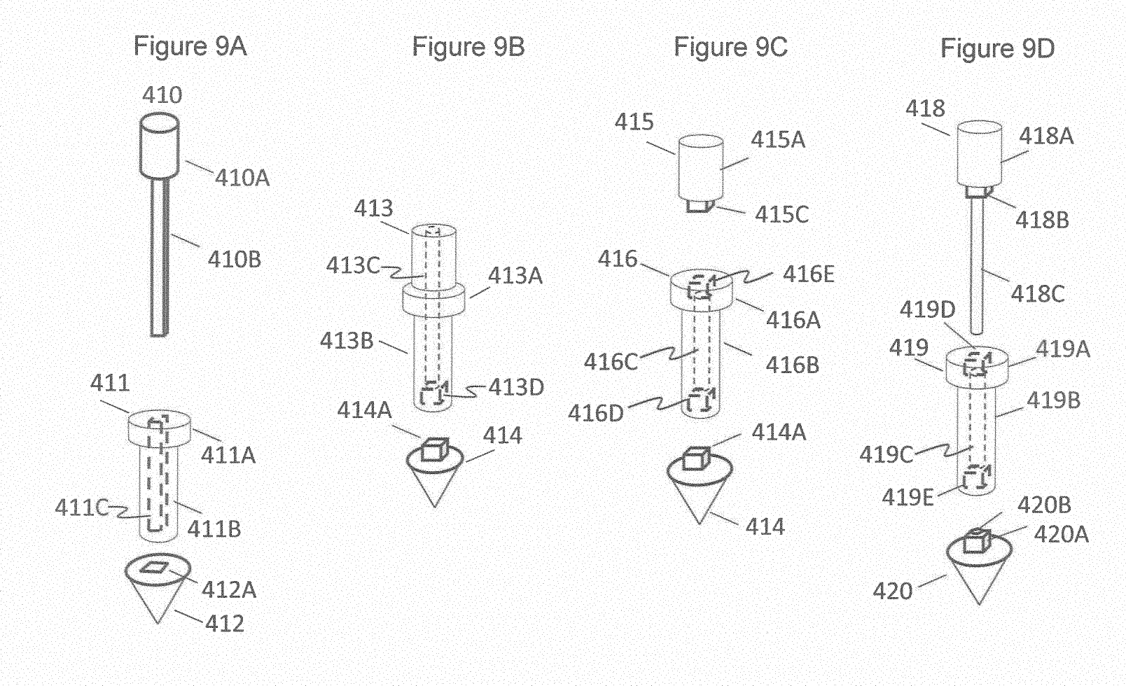

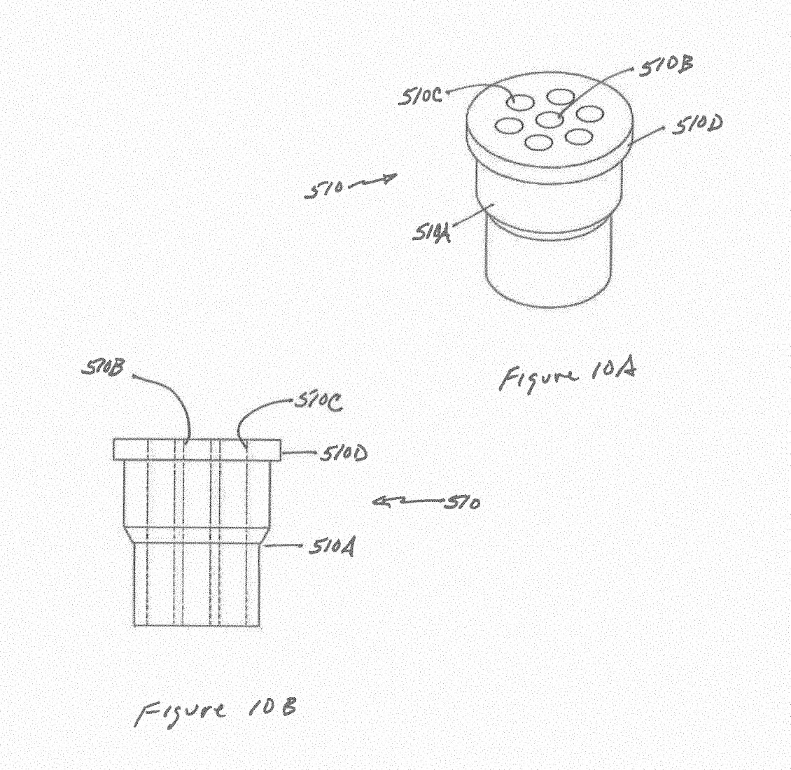

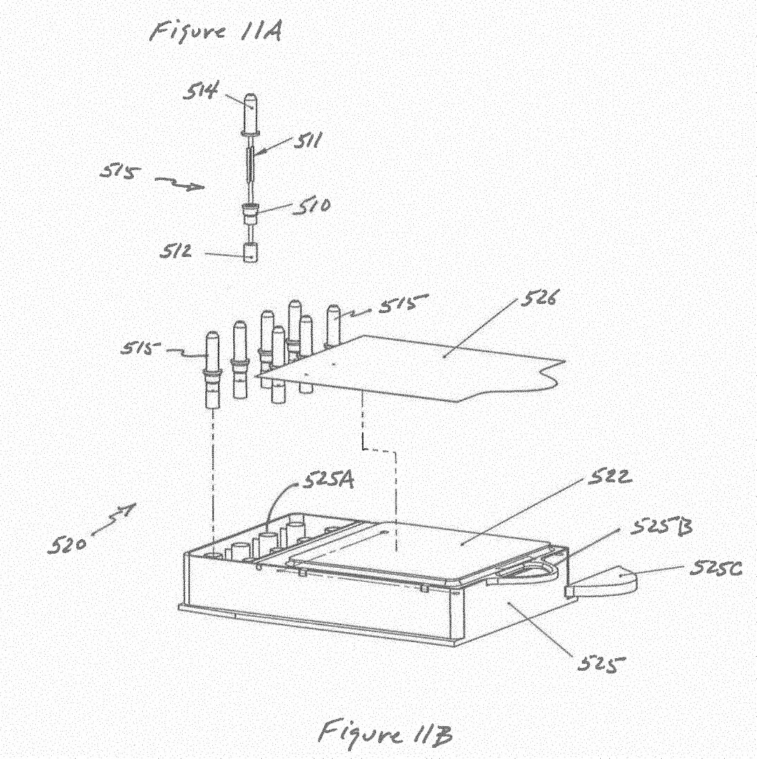

According to another aspect of the disclosure, a number of adapter and/or guide tube and drill bit configurations may be used for joining drill bits to guide tubes and for transmitting torque from a drill to a drill bit and/or guide tube without contact between the drill head and the drill bit and/or guide tube. Referring to FIG. 9A, an exploded perspective view of a configuration is illustrated in which an adapter 410 is receivable within a guide tube 411 and a drill bit 412 to allow transmission of torque from the adapter to the drill bit. In FIG. 9A, adapter 410 comprises a cylindrical drive knob 410A having a rod 410B extending outward therefrom. In the illustrative embodiment, rod 410B has a rectangular cross-sectional profile. Guide tube 411 comprises a generally cylindrical body 411B defining a central passage or lumen 411C extending therethrough. In the illustrative embodiment, passage 411C has a cross-sectional profile which mimics that of rod 410B but is sized to allow insertion of rod 410B therein. Guide tube 411 further comprises a flanged head 411A at one end thereof. Drill bit 412 is shape similar to drill bit 401 of FIG. 7A but has a cavity 412A extending at least partially into the interior thereof. In the illustrative embodiment, cavity 412A has a cross-sectional profile which mimics that of rod 410B but is sized to allow insertion of rod 4100B therein. Drill bit 412 is driven by attachment to rod 410B connected to drive knob 410A. In the use, the adapter 410, guide tube 411 and drill bit 412 may be preconfigured together, as similarly illustrated in FIGS. 3 and 4 with rod 410B disposed within passage 411C and cavity 412A. A drill chuck may grab drive knob 410A to pick up the assembly and position drill bit 412 over plate 108 to perform the drilling/welding procedure. After the guide 411 is welded in place, the drill chuck pulls the drive knob 410A in a retrograde direction, removing the rod 410B and allowing the bit 412 to fall away.

Referring to FIG. 9B, an exploded perspective view of a system configuration is illustrated in which guide tube 413 comprises cylindrical body 413B that extends above flange 413A and a passage 413C having a circular cross-sectional profile that opens at one end thereof into a rectangular shaped cavity 413D which is sized to receive stub extension 414A of drill bit 414 therein. The portion of guide tube 413B that extends above flange 413A is receivable within an adapter not shown, substantially similar to adapter 301 and attached to a drill, so that torque is transmitted from the adapter to the drill bit. In the use, the guide tube 413 and drill bit 414 may be preconfigured together. A drill fitted with the adapter picks up the assembly and positions drill bit 414 over plate 108 to perform the drilling/welding procedure. After the guide 413 is welded in place, the drill with adapter releases guide tube 413, leaving bit 414 to be removed manually.

Referring to FIG. 9C, an exploded perspective view of a system configuration is illustrated in which stub 415C of adapter 415 and stub 414A of a drill bit 414 are receivable within a guide tube 416 to allow transmission of torque from the adapter to the drill bit. Adapter 415 is implemented substantially as previously described but without a rod e.g., 410B extending outwardly therefrom. In the use, the adapter 415, guide tube 416 and drill bit 414 may be preconfigured together. A drill chuck grabs drive knob 415A to pick up the assembly and position drill bit 414 over plate 108 to perform the drilling/welding procedure. After the guide 416 is welded in place, the drill chuck pulls the drive knob 415A in a retrograde direction, leaving the bit 414 to be removed manually.

In FIG. 9D, adapter 418 comprises a cylindrical drive knob 418A having a rectangular stub extension 418B extending outwardly therefrom and a rod 418C extending outward from stub extension 418B. In the illustrative embodiment, rod 418C has a circular cross-sectional profile. Guide tube 419 comprises a generally cylindrical body 419B defining a central passage or lumen 419C extending therethrough. In the illustrative embodiment, passage 419C has a circular cross-sectional profile which mimics that of rod 418C but is sized to allow insertion of rod 418C therein. Guide tube 419 further comprises a flanged head 419A at one end thereof. Drill bit 420 may be implemented similar as described previously herein. Passage 419C opens into a rectangular shaped cavities 419D and 419E which are sized to receive stub extension 420A of drill bit 420 and stub extension 418B of adapter 418, respectively therein. Drill bit 420 is held into the guide tube 419 by rod 418B which fits into circular cavity 420B in the drill bit. Drill bit 420 is driven by attachment to guide 419. In the use, the adapter 418, guide tube 419, and drill bit 430 may be preconfigured together, with rod 418C disposed within passages 419C and 420B. A drill chuck grabs drive knob 418A to pick up the assembly and position drill bit 420 over plate 108 to perform the drilling/welding procedure. After the guide 419 is welded in place, the drill chuck pulls the drive knob 418A in a retrograde direction, removing the rod 418C and allowing the bit 420 to fall away.

According to another aspect of the disclosure, a guide tube includes multiple guide passages so that a single guide tube provides the option for multiple, clustered insertion points in a single area from a single guide tube. Referring to FIGS. 10A-B, a guide tube 510 comprises a generally cylindrical body 510A, having a plurality of sections with different cross-sectional diameters. Guide tube body 510A further defines a central needle guide passage or lumen 510B extending therethrough and a plurality of needle guide side passages 510C surrounding lumen 510B. In the illustrative embodiment, passages 510C are evenly spaced about passage 510B with the respective centers of passages 510C located on a circle radius measured from the center of passage 510B. In the illustrative embodiment, passages 510B-C have a cross-sectional profile which mimics that of rods 511 but is sized to allow insertion of rods 511 therein. Guide tube 510 further comprises a flanged head 511D at one end thereof defining an abrupt increase in diameter in comparison to the diameter of cylindrical body 510A. Guide tube 510 may be paired with a drill bit and adapter similar to the adapter/guide tube/drill bit systems described herein for joining drill bits to guide tubes and for transmitting torque from a drill to a drill bit and/or guide tube without contact between the drill head and the drill bit and/or guide tube.

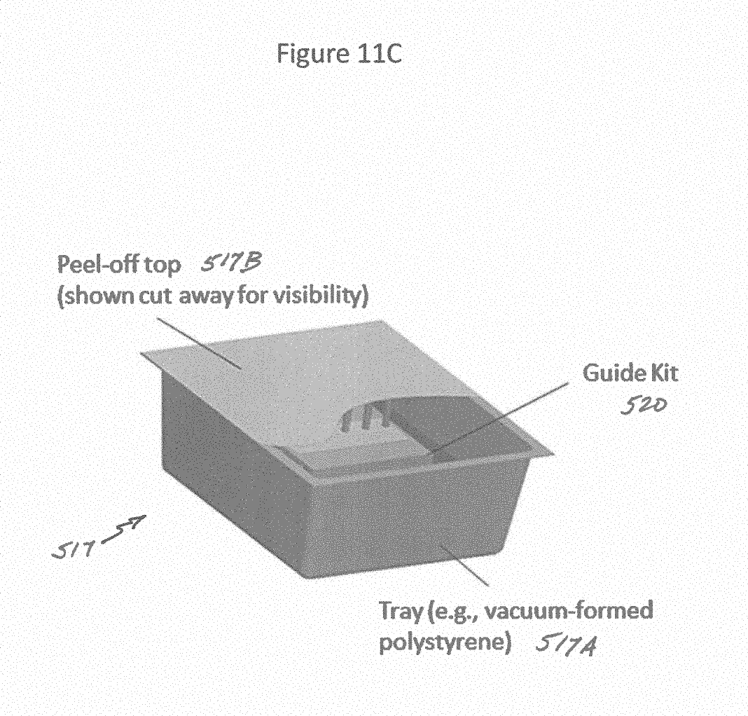

Referring to FIG. 11A, an exploded perspective view of a guide tube stack 515 is illustrated as comprising an adapter 514, guide tube 510, rods 511 and drill bit 512. A pair of rods 511 is receivable within any combination of passages 510B-C of a guide tube 510 and drill bit 512 to allow transmission of torque from the adapter 514 to the drill bit 512. Drill bit 512 maybe shape similar to drill bit 404 of FIG. 8A or drill bit 406 of FIG. 8B but has a central passage and at least one off-center passage at least partially extending therethrough and having cross-sectional passage profiles which mimic that of rods 510 but are sized to allow insertion of rods 510 therein. Drill bit 512 is driven by attachment to rods 511 which are, in turn, connected to adapter 514. In the use, the adapter 514, guide tube 511 and drill bit 512 may be preconfigured together into guide tube system 515, as similarly illustrated in FIG. 11B with rods 511 disposed within passages 511B-C and similar corresponding passages of drill bit 512. A drill chuck may grab adapter 514 to pick up the assembly and position drill bit 512 over plate 108 to perform the drilling/welding procedure. After the guide 510 is welded in place, the drill chuck pulls the adapter 514, in a retrograde direction, removing the rods 511 and allowing the drill bit 512 to fall away, in a procedure similar to that utilizing the other guide tube and drillbit combinations described herein.

FIG. 11B is a conceptual exploded view of a kit 520 comprising a tray 525 to which a plate 522 and a plurality of guide tube stack 515 may be removably secured. In the illustrative embodiment, tray 525 has a generally rectangular shape defining a plurality of interior segmented cavities, one of which defines a plurality of sockets 525A projecting outward therefrom and into which guide tube systems 515 may be removably received. In embodiments, either adapter cap 514 or drillbit 512 may be received into sockets 525A. Plate 522, as illustrated, has a generally rectangular shape with a plurality of clips about the peripheral edges thereof for securing to the perimeter edges of tray 525. In embodiments, tray 525 may have handle 525B and handle cover 525C to assist with handling of kit 520 in a sterile environment. Plate 522, may have the same construction and function as plate 108 described herein. An optional film 526 may be disposed adjacent to the surface of the plate 522. Film 522 is used to protect the surface of plate 522 from a non sterile environment.

In embodiments, any of drill bits 401-404, 406, 412, 414, 420 or 512 may be formed of Polyether Ether Ketone PEEK plastic, a colourless organic thermoplastic polymer in the polyaryletherketone PAEK family. PEEK plastic is a semicrystalline thermoplastic with excellent mechanical and chemical resistance properties that are retained to high temperatures. PEEK plastic melts at a relatively high temperature 343° C./649.4° F. compared to most other thermoplastics enabling any of drill bits to be formed or processed using injection moulding or extrusion methods. Any of drill bits 401-404, 406, 412, 414, 420 or 512 may also be formed of aluminum or stainless steel, or any material having a higher melting temperature than the plate 108 and which is magnetic resonance compatible.

In embodiments, any of adapter 410, 415, 420 and 514 may be formed of stainless steel or other rigid, sterilizable material. In embodiments, any of guide tubes 411, 413, 416, 419 and 510 may be formed of plastic, including, but not limited to, natural or synthetic resins which are rigid enough to transmit torque from the adapter to the drill bit but which have a lower melting point than the drill bit for fusing with plate 207 during the drilling/welding process.

Because the environment in the Guide Fabrication Machine (GFM) used to create the needle guide is likely to become contaminated, leading to contamination of the needle guides it produces, a need exists for a mechanism in which the needle guide can be produced without being exposed to an external environment.

According to another aspect of the disclosure, the needle guide kit 520, and its needle guide components, are completely enclosed in a sealed sterile cover. Prior to use, e.g., during shipping and storage, the kit 520 is contained inside an outer sterile package 517, shown in FIG. 11C. In one embodiment, the package 517 may comprise a vacuum-formed polystyrene tray 517A in which the kit 520 is disposed and retained with a peel-off top 517B. The kit 520 may be sterilized using gamma radiation after packaging.

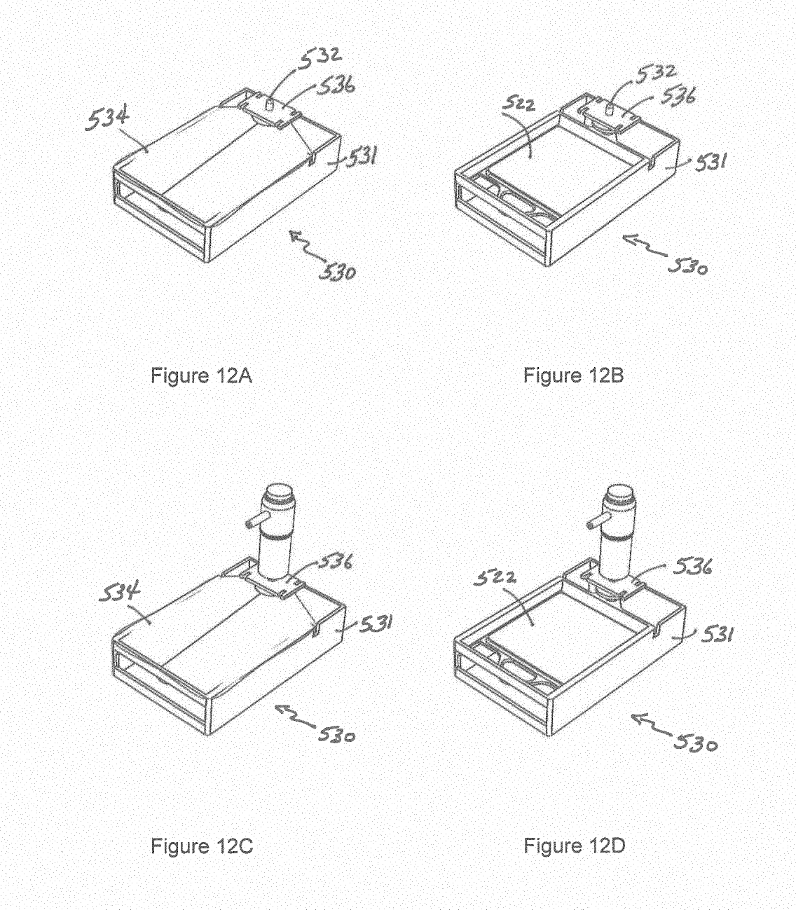

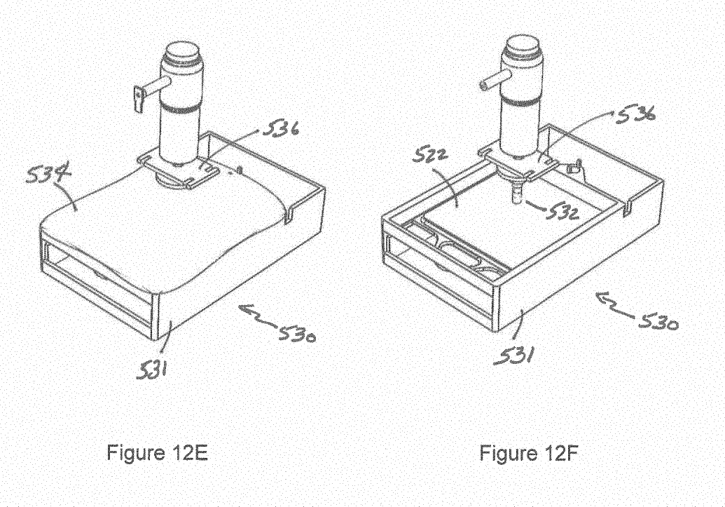

According to another aspect of the disclosure, the internal components of a needle guide kit are isolated from the outside environment during fabrication. In one embodiment, a needle guide kit 530 may comprise a tray 531 holding substantially the same components as kit 520 described herein but with only a single stack 532. In addition, a bag 534 is sealingly fixed over the top edge of tray 531 above the surface of plate 522. In addition, a peel away decal, not shown, similar to decal 557 described elsewhere herein may be disposed over the open end of tray 531 help isolate the plate 552 from the environment exterior to kit 530. Bag 534 has fixed thereto an interface 536 through which the stack 532 is movably disposed, as illustrated in FIGS. 12A-B. Interface 536 may comprise, in one embodiment, a rubber seal fixed directly adjacent the exterior surface of bag 534 and a rigid or semirigid holder disposed adjacent the rubber seal, both the rubber seal and holder having apertures to movably retain stack 532 therein. Bag 534 is dimensioned large enough so that interface 536 may be moved in three axes relative to the surface of plate 108 during the fabrication process and may be made from a thin flexible antimicrobial material. As such, bag 534 may be folded upon itself during transport and storage with the interior of kit 530 remaining sterile. FIGS. 12A-F illustrate conceptually the sterile fabrication process used to generate a custom needle guide from kit 530, with FIGS. 12B, 12D, 12F included only for comparison purposes to indicate the relative position of interface 536 relative to plate 522 during the process of placing the guide tube.

FIGS. 12A, 12C, and 12E depict the process for placing a guide tube at a desired location on a plate using the computer-controlled actuators of a guide fabrication machine. First, the drill head is positioned directly above the stack 532 and then lowered until the drill head is over the cap of stack 532, as illustrated in FIG. 12C. Next, an automatic chuck is activated, grabbing the cap and, therefore, the entire stack 532. The stack 532 is repositioned over the location where the guide tube is to be placed, as illustrated in FIG. 12E. This motion may be in two dimensions to cover the entire plate as needed, but is shown only along one axis in FIG. 12 for purposes of explanation. Note that as the drill moves the components of the stack, the flexible sterile barrier created by bag 534 deforms in three dimensions, as needed, to accommodate the motion and thus remains a sealed barrier throughout the production process. Next, the drill spins and lowers the stack. The drill bit heats the plate by friction, creating a hole as it passes through the plate. The drill bit may be made of PEEK, a high-temperature plastic that has a melting point well above that of the plate material (ABS). The interior cavity of the bit captures most of the plastic that is removed from the hole, and the remainder of the displaced plastic forms a rim around the hole. Friction drilling eliminates the formation of small debris particles that are produced in conventional drilling. After the hole is created, the spinning stack 532 continues advancing until the flange of the guide tube reaches the plate 522. Friction between the flange and the plate melt a thin layer of plastic on each welding the two surfaces together. The entire process, including drilling, welding, and cooling, may take only about 10 seconds. Once the guide tube has been welded into the plate, the drill is retracted. Because the drill chuck is gripping the cap, the cap and pins are pulled from the stack 532. The drill bit falls below the plate 522 into the tray 531.

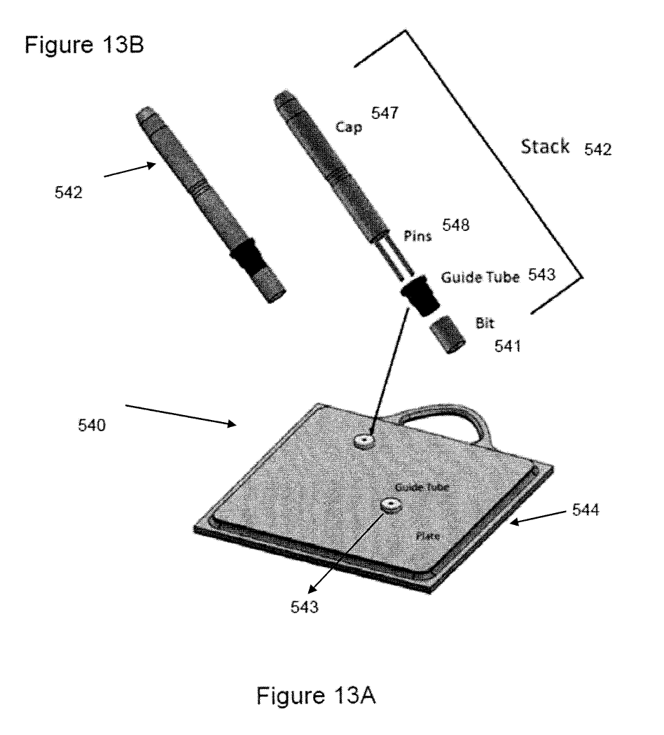

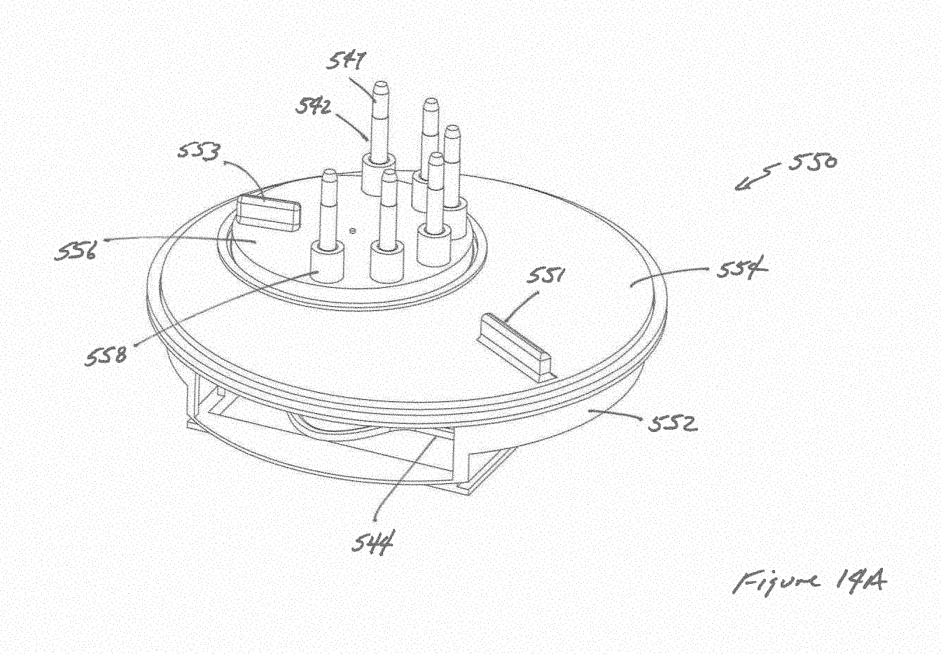

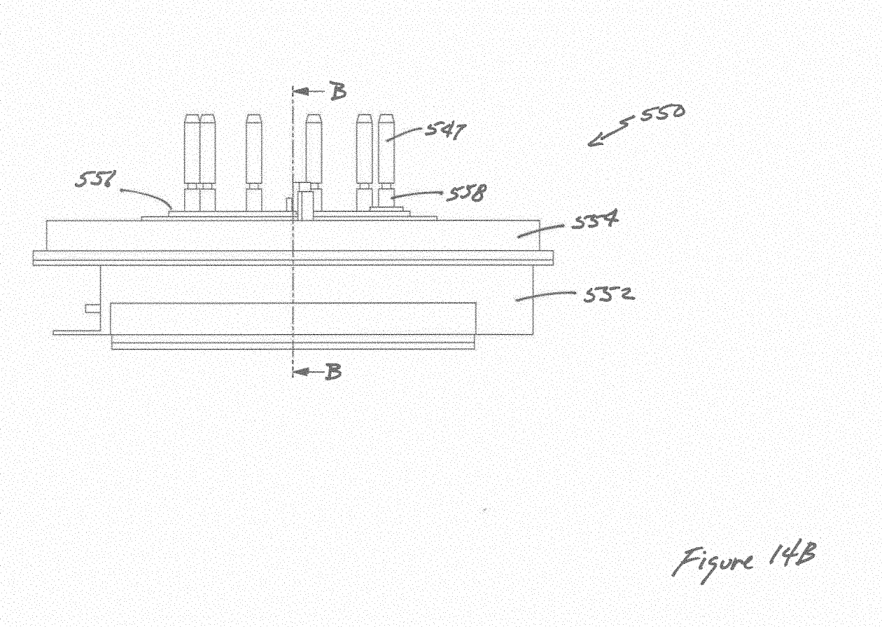

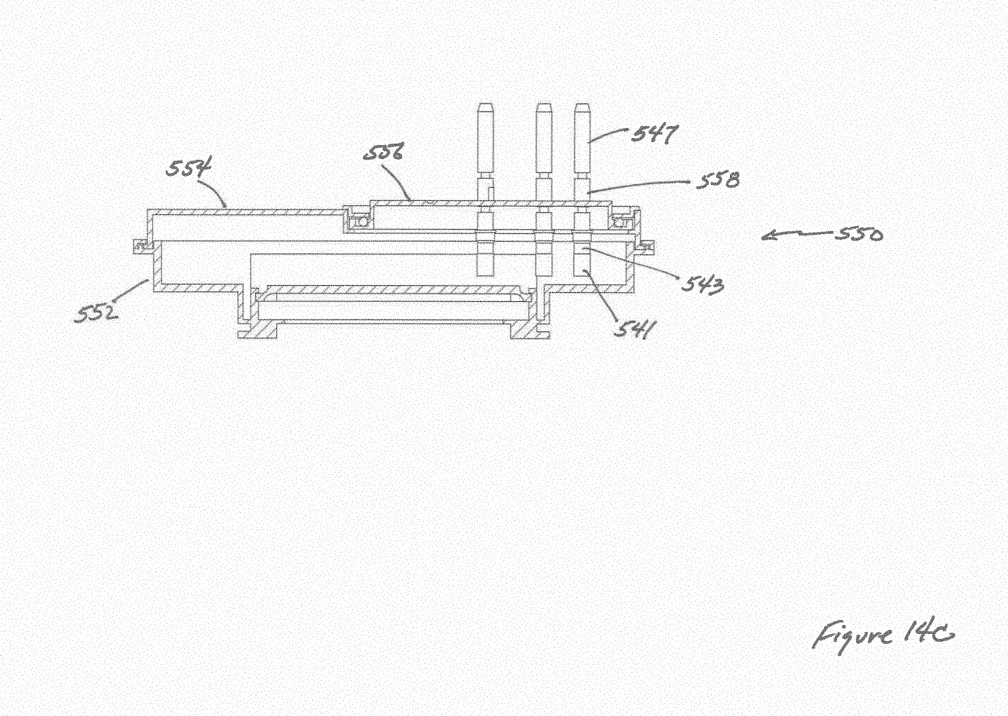

FIGS. 13A-B illustrate another embodiment a stack 542 for fabrication of a clean needle guide 540. The kit from which the needle guide 540 is made includes a plate 544 and one or more stacks 542, each consisting of a bit 541, a guide tube 543, a cap 547, and pins 548. The pins 548 are fixed to the cap 547 and pressed into the bit 541 and can transmit torque to the bit 541 and guide tube 543.

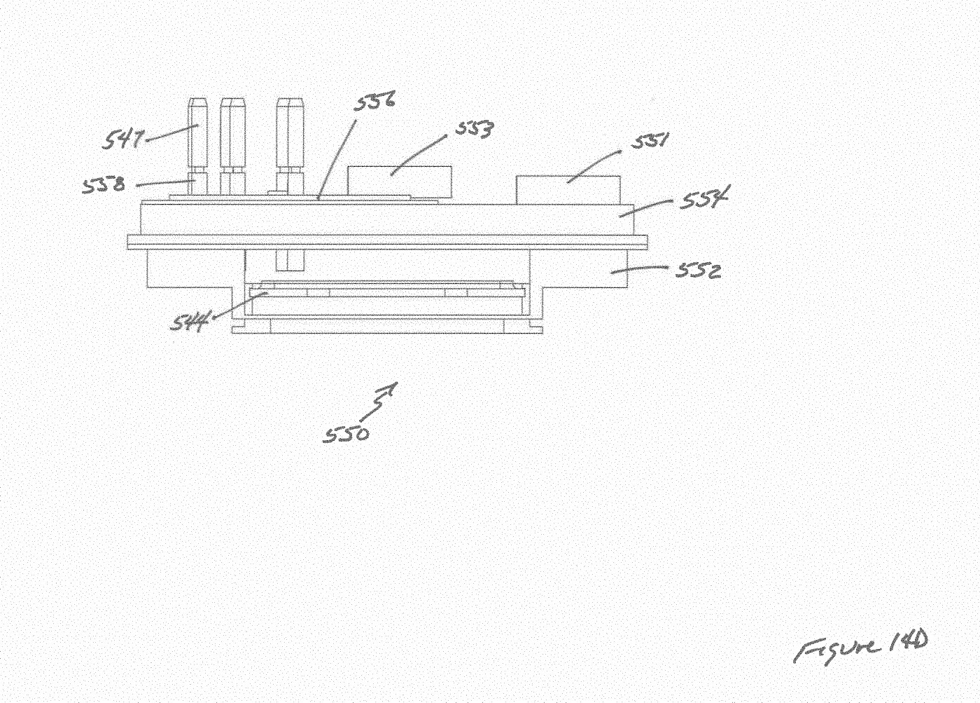

FIGS. 14A-D illustrate the construction of a needle guide fabrication kit 550, according to another aspect of the disclosure, in which the component parts of the kit necessary to construct a needle guide are completely contained inside a barrier, Friction drilling and welding during the needle guide fabrication process are accomplished by working through sealed bushings, allowing holes to be drilled and guide tubes welded into place without transferring contaminants across the barrier. In an illustrative embodiment, needle guide kit 550 comprises a substantially circular shaped base 552 defining a cavity and having an aperture along the perimeter thereof into which plate 544 may be slidably removed. Base 552 is enclosed with a cover 554 the rims of which form labyrinth seals 555. Allowing for rotational movement of cover 554 upon the application of force to a panel 551 projecting upward from the cover. A tour at 556 is rotationally secured through the labyrinth seals into the top of cover 544. Turn 556 may be rotated by the application of force to a Tarrant panel 553 projecting upward therefrom. A plurality of stacks 542 are retained within bushings 558 projecting upward from the top surface of turn 556 to allow interaction of caps 547 of each stack with a drill bit chuck or collect. A POA decal 557 may be used to temporarily seal plate 544 within the interior of base 552, as illustrated.

The turret 556 is retained in the rotary cove 554, which is retained by the base 552, so it is not possible to inadvertently lift either the turret or cover off of the kit, exposing the plate. As the cover 544 rotates, its peripheral edge slides along the top edge of the base 552. As the turret rotates, its outer edge slides against the cover. As shown in FIG. 14C, both the cover 554 and turret 556 use labyrinth seals 555 to prevent ingress of contaminants while allowing rotatation thereof. Labyrinth seals are not air-tight, however, they create a tortuous path through which contaminants will not pass readily without substantial force. To seal space from contaminants during the guide fabrication process, all rotating surfaces are sealed. The turret 556 and cover 554 are sealed with a flexible plastic skirt that rotates with the moving part and slides along the fixed part. The caps 547, which spin and slide for the drilling and friction welding process are sealed with a pair of elastomer rings, e.g., silicone o-rings, 559 which provide a high-reliability air-tight seal.

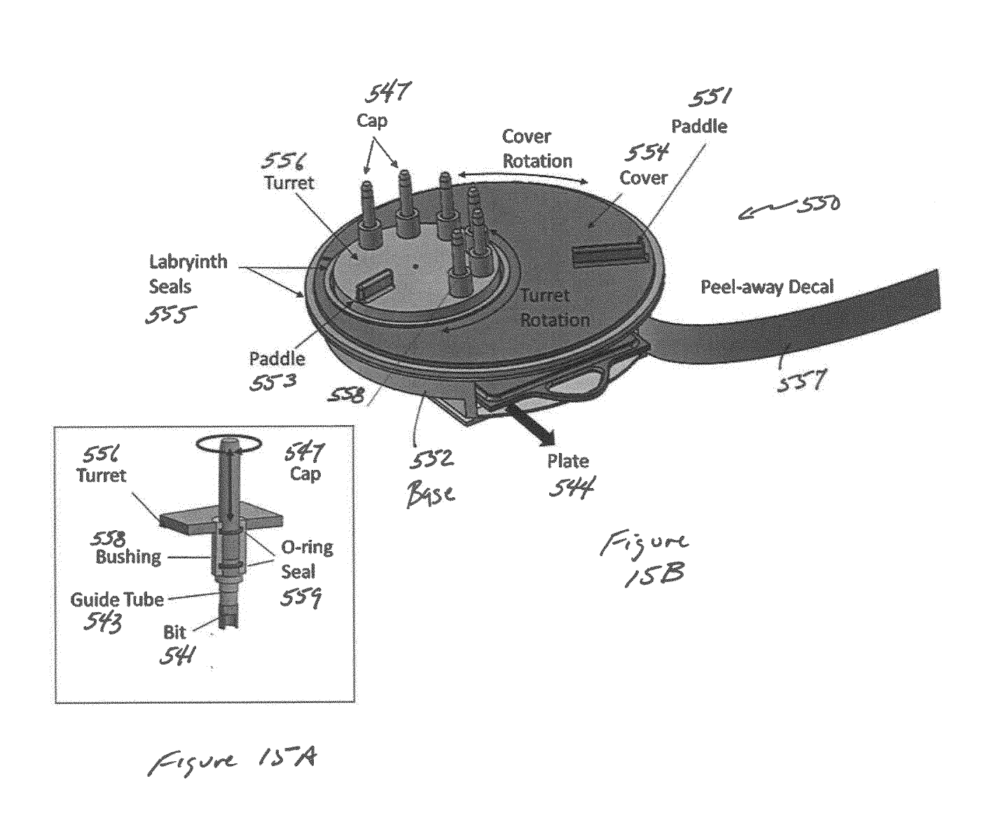

A clean environment is maintained during onsite guide manufacturing by inclusion of seals around the base/cover and cover/turret junctions, as well as around the caps where they pass through the turret. Note that the seals shown in FIG. 15A only need to maintain a clean barrier during fabrication of the guide and transport into the MRI room.

The caps 547 are extensions of the drill bits 541 and guide tubes 543 that protrude through seals 559 in the turret 556. The Guide Fabrication Machine (GFM) can grip the caps and drill just as with the previous design. The caps 547 are situated within a pair of o-ring seals which can allow the cap to rotate and slide while maintaining an air-tight seal. The o-rings may be made of silicone, buna nitrile, or other elastomer. The ring cross section, shown on the bottom of FIG. 3, is deformed when the ring is pressed between two surfaces. This is the same type of sliding seal used in many syringes.

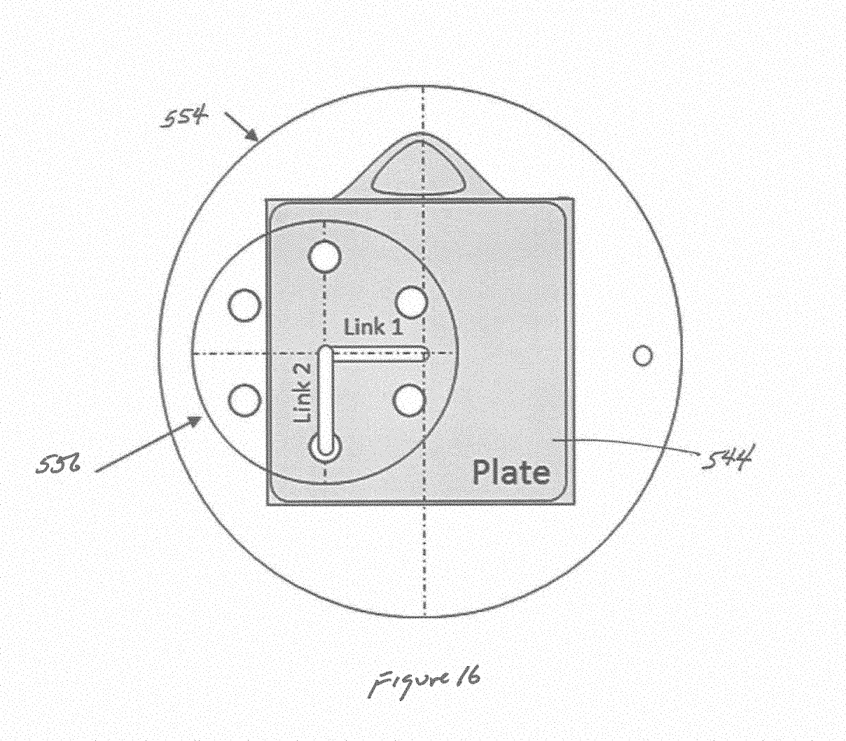

FIG. 16 illustrates conceptually the kinematics of rotary design relative to plate 544, illustrated in phantom to indicate that it is beneath turret 556 and cover 554. The two rotating pieces, turret 556 and cover 554, are equivalent to two virtual links (or line segments). If the links are of equal length (the bit circle crosses the center of the cover) and the sum of the lengths Link1+Link2 is at least the distance from the cover center to the furthest point on the cover, the bits can be positioned over any point on the plate 544. By rotating the turret 556 and cover 554 independently, any of the bits 541 can be positioned over any point on the plate 544, allowing placement of up to six guide tubes 543 at desired positions on the plate 544. This this process requires that the circle containing the bits passes through the center of rotation of the cover, and that the sum of the distances from the cover center-of-rotation to the turret center-of-rotation and the radius of the bit circle are at least as large as the distance from the plate center to the plate corner.

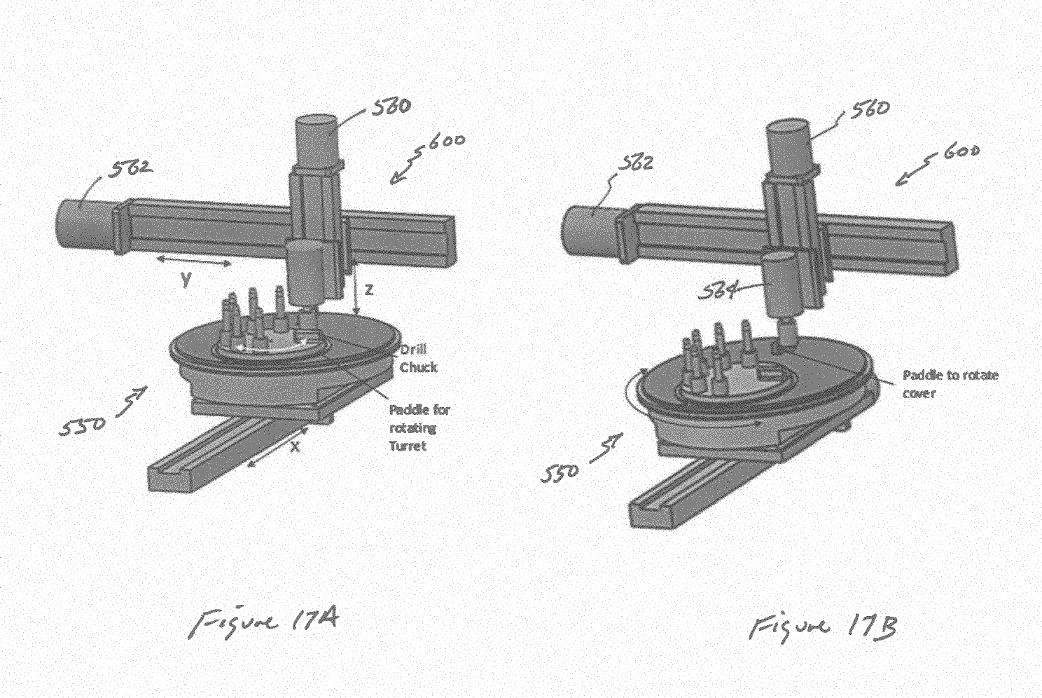

The xyz traverse in the GFM 600 comprises three robot stages that can move in coordination on, for example, circular paths. To position a drill bit and guide tube at a desired location, the xyz stage(s) push the paddles 553 and 551 with the drill chuck to rotate the turret 556 and rotary cover 554, as shown in FIG. 17A-B. In the FIG. 17A, the drill collet pushes paddle 551 and rotates the cover 554. In the FIG. 17B, the drill collet pushes paddle 553 and rotates the turret 556.

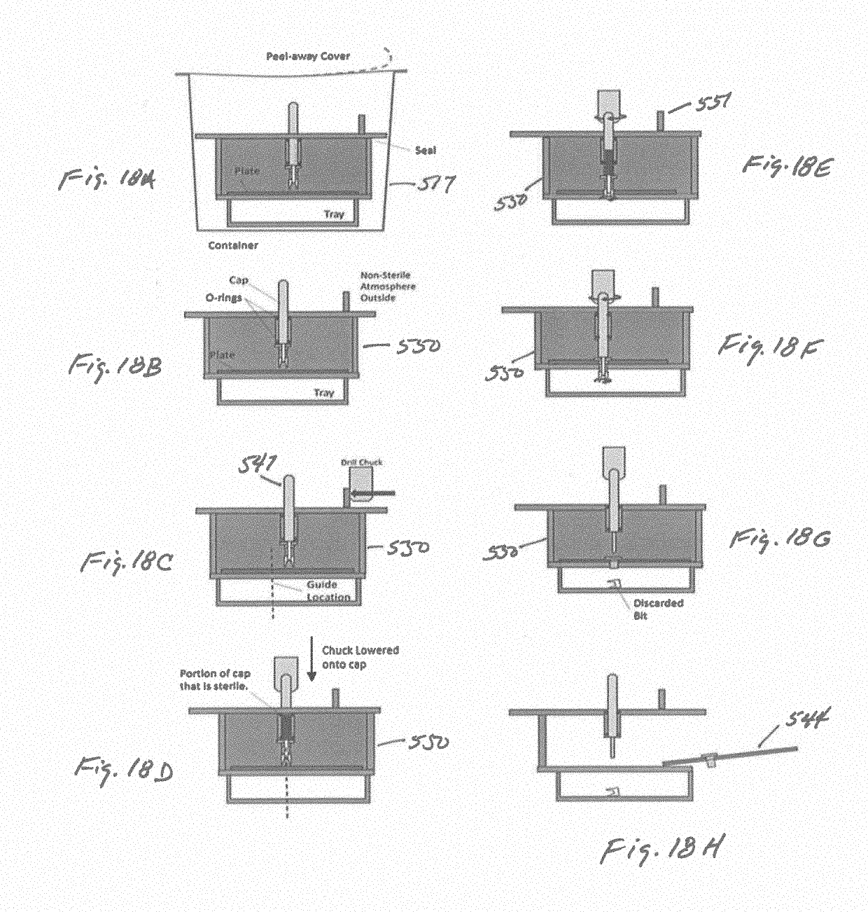

FIGS. 18A-H illustrate conceptually the process sequence by which the machine 600 is used to generate a custom needle guide from a kit 550 placing a guide tube 43 at a desired location on plate 544 with drilling and welding accomplished by working through sealed bushings 558. In an illustrative sequence of images in FIGS. 18A-H, assume that the cover 554 and turret 556 rotate 360 degrees. All steps are performed by the computer-controlled X, Y and Z actuators 560, 562 and 564, respectively, in the Guide Fabrication Machine (GFM) 600.

The kit 550 is shipped inside an outer sterile package 517. Prior to use, the kit 517 is removed from its packaging, as illustrated in FIG. 18A. The kit 517 is placed in the GFM 600 as illustrated in FIG. 18B. The drill is lowered beside the cover paddle 551 and uses the cover paddle to rotationally move the cover 554, and turret paddle 553 to rotationally move the current 556, to position the stack 542 over the desired guide location on plate 544 as illustrated in FIG. 18C. Next, the drill chuck is repositioned over the stack and lowered over the cap 547 thereof. Then, the automatic chuck is activated, grabbing the cap 547, as illustrated in FIG. 18D. The drill spins and lowers the bit 541, as illustrated in FIG. 18E-F. The drill bit 541 heats the plate 544 by friction, creating a hole as it passes through the plate 544. After the hole is created, the spinning stack 542 continues advancing until the flange of the guide tube 543 reaches the plate 108. Friction between the flange and the plate 544 melt a thin layer of plastic on each. Once the guide tube 543 has been welded into the plate 544, the drill is retracted. Because the chuck is gripping the cap, the cap 547 and pins 548 are pulled from the stack 542 The drill bit 544 falls below the plate into the tray. The foregoing steps are repeated for each guide tube 543 inserted into plate 544.

In clinical use, the kit 550 will be contained in a sealed outer package 517. At the start of the procedure, the technician will open the outer package, remove the kit 550, and place it in the guide fabrication machine 600. The barrier prevents contamination of the interior components. Once the custom needle guide has been fabricated, the kit is removed from the machine. The barrier is still intact and the components inside are clean, though the outside of the kit is not clean. The kit is transported to the procedure room. When the radiologist is ready to install the needle guide in the frame and perform the biopsy, the technician removes the adhesive cover 557 from the kit 550, and the the radiologist, with sterile gloved hands, will remove the custom fabricated needle guide for use. The GFM 600 will typically be located in a room adjacent to the MRI procedure room. The machine will be maintained to be clean but will not be sterile. The primary barrier around the kit and the seals as described help isolate the needle guide components from the time the package is opened until the decal 557 is peeled back for the radiologist to remove the plate 544.

The reader will appreciate that the disclosed fabrication process includes a barrier that fully encloses the parts of the needle guide kit, with friction drilling and welding accomplished by working through sealed bushings.

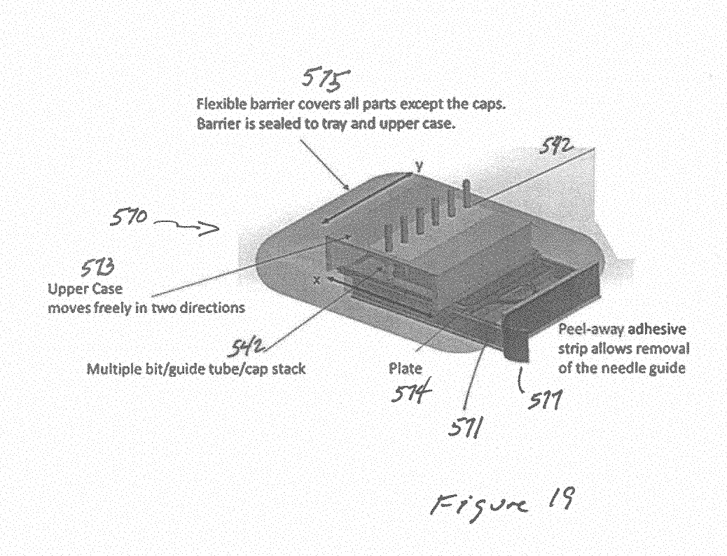

FIG. 19 illustrates another kit 570 with flexible barriers which allow a custom fabricated needle guide to remain completely isolated from airborne and surface contaminants in the exterior environment until it is removed from the outer package in the procedure room. In one embodiment, a needle guide kit 570 may comprise a tray 571 holding a plate 574 in an interior surface therein. Slidably fixed over lower tray 571 is an upper tray 573 which is slidable in both the X and Y axis using, for example, slidable tracks. Fixed to the top of upper tray 573 is one or more stacks 542 as previously described a flexible barrier 575 is disposed about the tray 571 and upper tray 573 and is sealed to the top of uppercase 573 to allow caps 547 of stacks 542 two protrude there from. Flexible barrier 575 is further sealed about an open end of lower tray 571 to allow for removal of plate 574 following the fabrication process. A peel away decal 577 may be used to cover open end of lower tray 571 prior to completion of the fabrication process. Flexible barrier 575 is dimensioned large enough so that upper tray 573 may be moved in along the X and Y axes relative to the surface of plate 574 during the fabrication process and may be made from a thin flexible antimicrobial material. The stack 542 are disposed in bushings, identical to bushings 558 of kit 550. The bits 541 are all kept in an upper tray 573 as it is translated translates in two directions. The xyz stage of the GFM 600 grips the caps 547 of stacks 542 using the automated chuck and uses them to move the upper tray 573 until a stack is over the desired location on plate 574.

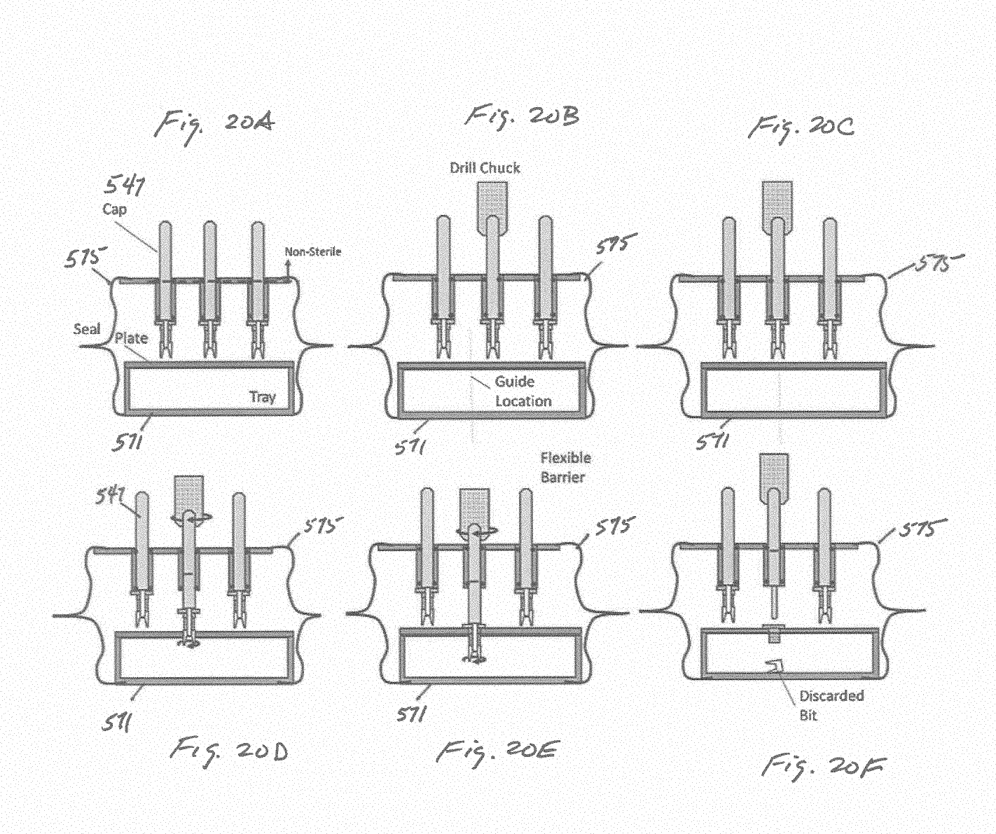

FIGS. 20A-F depict the process for placing a guide tube at a desired location on a plate 574 using kit 570 which was a guy of simplicity do not show the tracks fixing lower tray 571 two upper tray 573. All steps are performed by the computer-controlled actuators in the guide fabrication machine 600 in a manner substantially similar to that described with reference to kit 530 and FIG. 12. The drill is lowered until is over the cap 547. Then, the automatic chuck is activated, grabbing the cap and thus the entire stackv 542. The stack is repositioned over the location where the guide tube 543 is to be placed. This motion is in two dimensions to cover the entire plate 574 as needed, but is shown here in only one axis for purposes of explanation. Note that as the drill moves the components of the stack, the flexible sterile barrier 575 deforms, as needed to accommodate the motion and thus remains a sealed barrier throughout the fabrication process. The drill spins and lowers the stack 542. The drill bit heats the plate 574 by friction, creating a hole as it passes through the plate. After the hole is created, the spinning stack continues advancing until the flange of the guide tube reaches the plate. Friction between the flange and the plate melt a thin layer of plastic on each. The drill stops, allowing the parts to cool. The guide tube has been friction-welded into the plate. The entire process, including drilling, welding, and cooling, takes about 10 seconds. Once the guide tube has been welded into the plate, the drill is retracted. Because the chuck is gripping the cap, the cap and pins are pulled from the stack. The drill bit falls below the plate into the tray.

In other embodiments, the disclosed apparatus and techniques can be extended for use in other MRI-guided procedures that require biopsies or treatments for prostate cancer.

It will be obvious to those recently skilled in the art that modifications to the apparatus and process disclosed here in may occur, including substitution of various component values or nodes of connection, without parting from the true spirit and scope of the disclosure.

What is claimed is:

1. A kit for manufacturing a needle guide comprising:
a plurality of cylindrical guide tubes, each guide tube defining at least one needle guide passage extending therethrough,
a plurality drill bits, each drill bit associated with one of the plurality of guide tubes.

2. The kit of claim 1 wherein one of the plurality of drill bits is removably securable to one of the plurality of guide tubes at a first end thereof.

3. The kit of claim 1 wherein at least one of the plurality of guide tubes defines multiple needle guide passage extending therethrough.

4. The kit of claim 1 further comprising at least one adapter for coupling one of the plurality of guide tubes to a motion source.

5. The kit of claim 4 further comprising a plurality of adapters, each adapter associated with one of the plurality of guide tubes and configured for coupling the one guide tubes to a motion source.

6. The kit of claim 4 wherein the at least one adapter is attachable of one of the plurality of guide tubes at a first end thereof.

7. The kit of claim 4 wherein the at least one adapter is attachable of one of the plurality of drill bits at a first end thereof.

8. The kit of claim 1 further comprising a housing for retaining the plurality of guide tubes and the plurality of drill bits.

9. The kit of claim 1 further comprising a planar plate formed of a material having a lower melting point than the plurality of drill bits.

10. In combination:
a cylindrical guide tube defining an interior needle guide passage extending therethrough,
a drill bit coupled to the guide tube at a first end thereof, and
an adapter for coupling one of the drill bit and guide tube to a source of motion

11. The combination of claim 10 further comprising a housing surrounding and maintaining the guide tube and drill bit in a sterile condition.

12. The combination of claim 10 further comprising a planar plate formed of a material having a lower melting point than the drill bit.

13. The combination of claim 13 wherein the housing is defined by a flexible wall which allows the guide tube and drill bit to be positioned relative to the planar plate while maintaining the sterile condition of the guide tube and plate.

14. A needle guide for use during a biopsy procedure comprising a cylindrical guide tube body defining a plurality of needle guide passages extending therethrough and having a first end defined for coupling to a drill bit and a second end defined for coupling with an adapter.

15. The needle guide of claim 14 wherein the cylindrical guide tube body comprises a first portion having a first diameter and a second portion having a second diameter greater than the first diameter.

16. A kit for preparing a needle guide for biopsy procedures comprising:
a cylindrical guide tube defining an interior needle guide passage extending therethrough,
a drill bit coupled to the guide tube at a first end thereof,
a planar plate formed of a material having a lower melting point than the drill bit; and
an encapsulating structure defining a selectively accessible interior space in which the guide tube and planar plate are disposed.

17. The kit of claim 16 wherein the encapsulating structure is defined by a wall which is at least partially movable relative to another section of the encapsulating structure and which allows the guide tube to be re-positioned along three axes relative to the planar plate while remaining within the interior space of the encapsulating structure.

18. The kit of claim 17 wherein the wall is rotatably movable relative to said another section of the encapsulating structure.

19. The kit of claim 17 wherein the wall is flexibly movable relative to said another section of the encapsulating structure.

20. The kit of claim 17 further comprising:
an adapter for coupling one of the drill bit and guide tube to a source of motion, the adapter being disposed at least partially exterior of the encapsulating structure.
